(19) United States
(12) Patent Application Publication
(10) Pub. No.: US 2017/0135423 A1
(43) Pub. Date: May 18, 2017

(54) Diaper Cover Leggings with High Volume Wet Zone Protection

(71) Applicant: Michael Nau, Belton, TX (US)

(72) Inventor: Michael Nau, Belton, TX (US)

(21) Appl. No.: 15/130,533

(22) Filed: Apr. 15, 2016

Related U.S. Application Data
(60) Provisional application No. 62/256,268, filed on Nov. 17, 2015.

(51) Int. Cl. A41D 17/02; A41D 27/20; A41D 31/02; A61F 13/84

(52) U.S. Cl. CPC A41D 17/02; A61F 13/84; A41D 27/20; A41D 31/02; A41D 2400/62

(57) ABSTRACT

A pair of diaper cover leggings with high volume wet zone protection includes a waistband, a bifurcated legged outer layer, and an inner layer. The bifurcated legged outer layer and the wet-zone inner layer are connected to the waistband and downwardly extend from the waistband. The bifurcated legged outer layer extends up to a lower left-leg hem and a lower right-leg hem in order to complete the external layer of the diaper cover leggings. The inner layer extends up to an outer crotch section of the bifurcated legged outer layer and connected to the outer crotch section. As a result, the bifurcated legged outer layer and the inner layer are able to form the high volume wet zone to improve the absorbency. Additionally, the bifurcated legged outer layer transitions into a single layered comfort zone beyond the high volume wet zone.

The current application claims a priority to the U.S. Provisional Patent application Ser. No. 62/256,268 filed on Nov. 17, 2015.

### FIELD OF THE INVENTION

The present invention relates generally to leggings. More specifically, the present invention is a multi-layered diaper cover and a pair of leggings which comprises a high volume wet zone to maximize absorbency. The present invention is designed to be worn over diapers and to provide a comfortable protective covering for the wearer.

### BACKGROUND OF THE INVENTION

Many parents choose to use cloth diapers over disposable diapers for a number of reasons, ranging from the environmental impact of disposable diapers to the health concerns of the synthetic materials used in disposable diapers. Cloth diapers require the use of a diaper cover or a pair of diaper cover leggings to reduce and eliminate leakage, as cloth diapers are not as absorbent as disposable diapers. Typical diaper covers generally utilize plastic, polyurethane laminate or other synthetic materials, which have a negative impact on the environment and can potentially be harmful to the infant's skin or health. Although diaper cover leggings exist today, the existing diaper cover leggings are not able to efficiently function with cloth diapers. For example, the existing diaper cover leggings are constructed to have a singular structure. Meaning, they are entirely constructed or knitted to be a single layered structure or double layered structure. In this regard, the existing doubled layered diaper cover leggings are either too bulky or too heavy. On the other hand, the existing single layered diaper cover leggings do not provide the protection and absorbency necessary to function properly as diaper covers.

It is an objective of the present invention to provide a pair of diaper cover leggings which improves upon the absorbency and comfort of diaper covers and diaper cover leggings that exist today. The present invention comprises a high volume wet zone that eliminates any chances of leakage from the infant's cloth diaper. The high volume wet zone also transitions into a single layered comfort zone, providing a more lightweight section for the wearer. The lightweight single layered comfort zone provides a more breathable and flexible area, maximizing the comfort for the wearer. Additionally, the present invention comprises various components to simplifies the wearing of the diaper cover leggings, as well as to prevent the diaper cover leggings from shifting when worn. Therefore, the present invention provides an improved pair of diaper cover leggings that maximizes the absorbency and comfort for the wearer.

### BRIEF DESCRIPTION OF THE DRAWINGS

FIG. 1 is a front view of the present invention showing the waistband, the bifurcated legged outer layer, and the inner layer.

FIG. 2 is a front view of the present invention without the left drawstring and the right drawstring in order to show the drawstring opening.

FIG. 3 is a front view of the present invention, wherein the left drawstring and the right drawstring are traversed through the drawstring opening.

FIG. 4 is a front view of the present invention showing the components of the inner layer.

FIG. 5 is a left side view of the present invention.

FIG. 6 is a rear view of the present invention.

FIG. 7 is a top view of the present invention.

FIG. 8 is a front view of the present invention, wherein the high volume wet zone entirely covers the diaper.

### DETAIL DESCRIPTIONS OF THE INVENTION

All illustrations of the drawings are for the purpose of describing selected versions of the present invention and are not intended to limit the scope of the present invention.

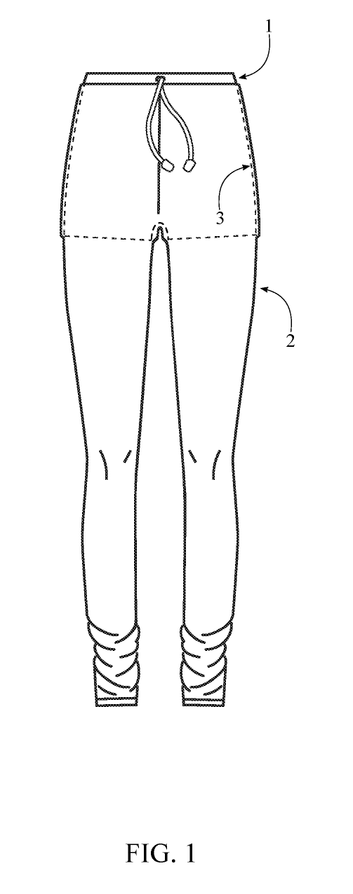

The present invention is a pair of diaper cover leggings that is meant to be worn over a cloth diaper. The present invention comprises a high volume wet zone and a comfort zone, which provide a significantly more absorbent and comfort protective covering for cloth diapers, compared to similar existing diaper covers. When worn properly, the high volume wet zone entirely covers the cloth diaper, protecting the wearer from any leaks. The present invention improves upon the existing inventions by providing a more comfortable and functional pair of diaper cover leggings. In reference to FIG. 1, the present invention comprises a waistband 1, a bifurcated legged outer layer 2, and an inner layer 3. The base structure of the present invention resembles a typical pair of leggings that includes a left leg hole, a right leg hole, and a waistband opening. In reference to the preferred embodiment, the waistband 1 secures the present invention to the waist of the wearer. The bifurcated legged outer layer 2 and the inner layer 3 collectively form a double layered high volume wet zone within the present invention. Additionally, the bifurcated legged outer layer 2 forms a single layered comfort zone that is positioned below the double layered high volume wet zone.

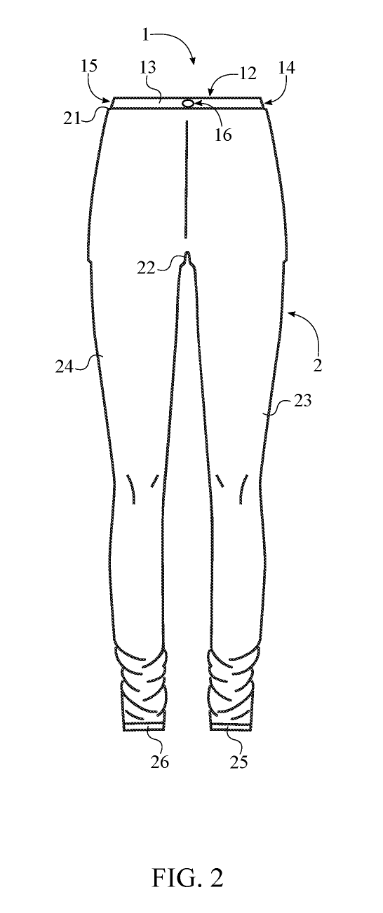

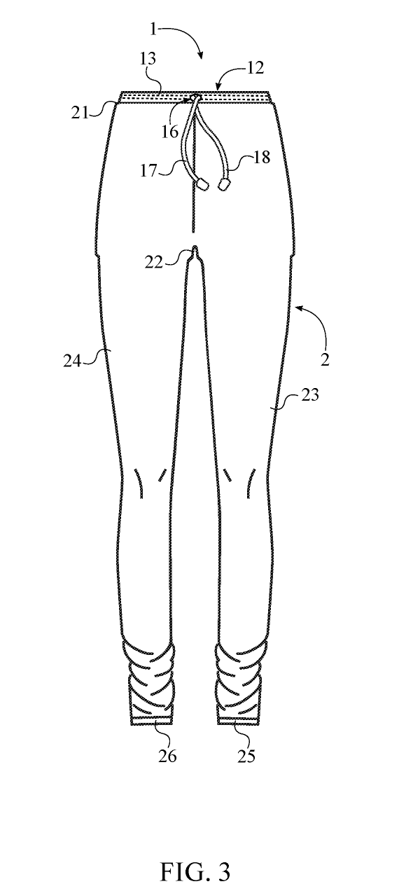

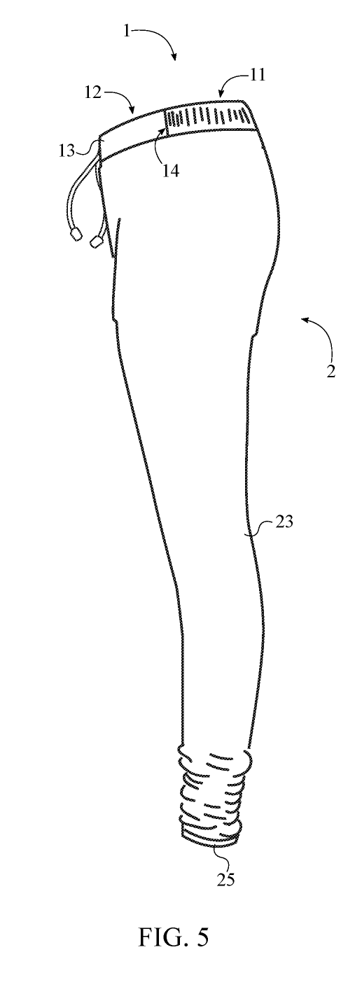

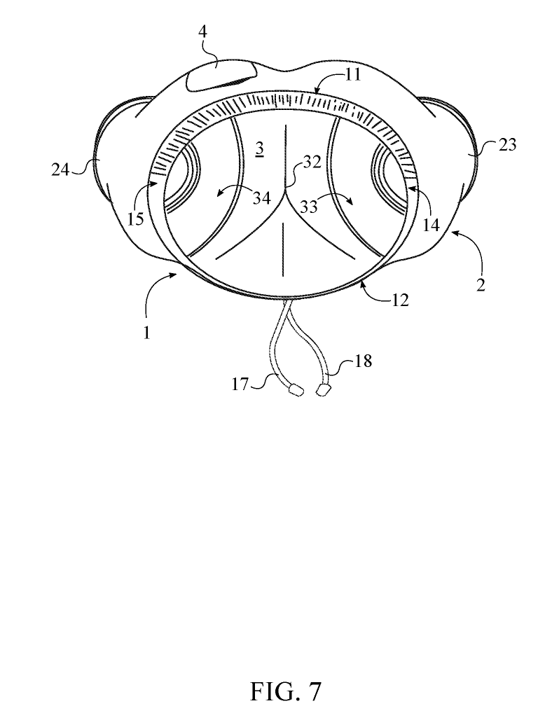

The waistband 1 allows the present invention to fit and accommodate a wide range of waist sizes. In reference to FIG. 5 and FIG. 7, the waistband 1 comprises an elastic rear portion 11 and a drawstring front portion 12. The elastic rear portion 11 and the drawstring front portion 12 are continuously connected to each other in order to form the waistband 1. The elastic rear portion 11, also known as the rear half of the waistband 1 or rear half circumference of the waistband 1, is constructed to have elastic properties. The elasticity of the elastic rear portion 11 allows the wearer to easily adjust the present invention when worn. In other words, the elasticity of the elastic rear portion 11 allows the user to stretch the waistband 1 for easy adjustments without having to utilize the drawstring front portion 12. In reference to FIG. 2-3 and FIG. 7, the drawstring front portion 12 comprises a channel body 13, a left drawstring 17, and a right drawstring 18. More specifically, a left end 14 and a right end 15 of the channel body 13 are connected to either end of the elastic rear portion 11 to complete the waistband 1. A drawstring opening 16 of the channel body 13 is centrally positioned in between the left end 14 and the right end 15 so that the left drawstring 17 and the right drawstring 18 can be traversed through the drawstring opening 16. In reference to FIG. 2-3, the left drawstring 17 is connected to the left end 14 in order to secure the left drawstring 17 to the drawstring front portion 12. The left drawstring 17 also extends along the channel body 13 and through the drawstring opening 16 so that a free end of the left drawstring 17 can be extended beyond the drawstring opening 16. Similarly, the right drawstring 18 is connected to the right end 15, resulting a secure connection between the right drawstring 18 and the drawstring front portion 12. The right drawstring 18 also extends along the channel body 13 and through the drawstring opening 16 so that a free end of the right drawstring 18 can be extended beyond the drawstring opening 16. As a result, the left drawstring 17 and the right drawstring 18 can constrict the drawstring front portion 12 and tie off to secure the waistband 1 around the waist of the wearer. The dual-waistband configuration, the elastic rear portion 11 and the drawstring front portion 12, provides the adjustability of a traditional drawstring-waistband while also providing the comfort of a traditional elastic-waistband.

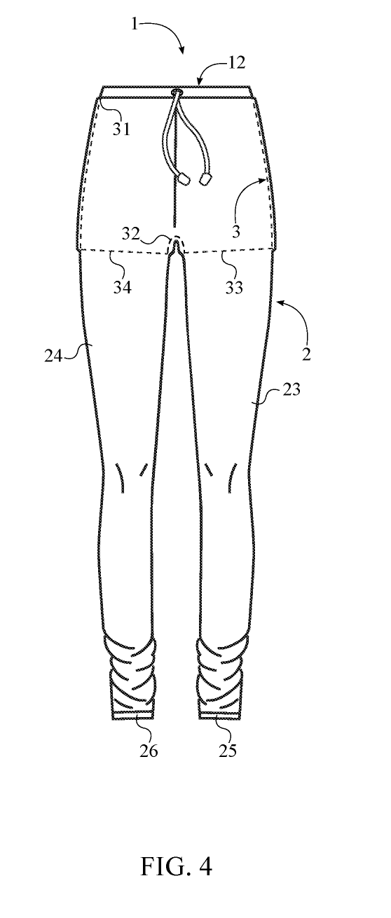

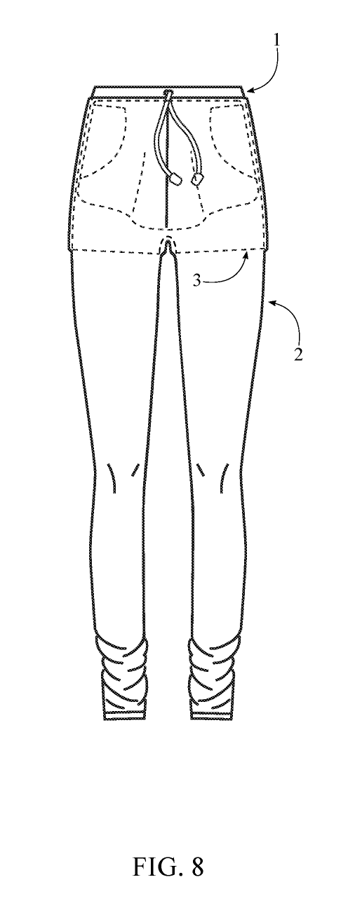

The bifurcated legged outer layer 2 provides an external surface area for the present invention and comprises a top outer edge 21, an outer crotch section 22, a left leg sleeve 23, and a right leg sleeve 24. In reference to FIG. 2 and FIG. 4, the top outer edge 21 is perimetrically connected around the waistband 1 so that the outer crotch section 22, the left leg sleeve 23, and the right leg sleeve 24 can downwardly extend from the waistband 1. The inner layer 3 comprises a top inner edge 31 and an inner crotch section 32. Similar to the bifurcated legged outer layer 2, the top inner edge 31 is perimetrically connected around the waistband 1 so that the inner crotch section 32 can downwardly extend from the waistband 1 to the outer crotch section 22. The inner crotch section 32 is perimetrically connected to the outer crotch section 22 so that the double layered high volume wet zone can be formed within the present invention. More specifically, the top inner edge 31 is adjacently positioned with the top outer edge 21 and perimetrically connected to the waistband 1. The top inner edge 31 and the top outer edge 21 delineate a top end of the double layered high volume wet zone. The inner crotch section 32 comprises an upper left-leg opening 33 and an upper right-leg opening 34. In order to delineate a bottom end of the double layered high volume wet zone, the upper left-leg opening 33 and the upper right-leg opening 34 are connected within the left leg sleeve 23 and the right leg sleeve 24, respectively. As a result, the double layered high volume wet zone is extended from the waistband 1 to the upper left-leg opening 33 and the upper right-leg opening 34. In other words, the double layered high volume wet zone spans from the waistband 1 to just below the outer crotch section 22. The double layered high volume wet zone preferably ends 1-2.5 inches below the crotch section 22 and maximizes the absorbency of the area which makes contact with the cloth diaper. In this regard, any leakage from within the cloth diaper is absorbed and held within the double layered high volume wet zone. The double layered high volume wet zone entirely covers the diaper when worn, as shown in FIG. 8.

The bifurcated legged outer layer 2 further comprises a lower left-leg hem 25 and a lower right-leg hem 26. In reference to FIG. 4, the left leg sleeve 23 extends from the waistband 1 to the lower left-leg hem 25, defining a left half of the bifurcated legged outer layer 2. The right leg sleeve 24 extends from the waistband 1 to the lower right-leg hem 26, defining a right half of the bifurcated legged outer layer 2. Since the double layered high volume wet zone is defined from the waistband 1 to the upper left-leg opening 33 and the upper right-leg opening 34, the single layered comfort zone is formed from the upper left-leg opening 33 and the upper right-leg opening 34. More specifically, the single layered comfort zone extends from the upper left-leg opening 33 to the lower left-leg hem 25 along the left leg sleeve 23 and from the upper right-leg opening 34 to the lower right-leg hem 26 along the right leg sleeve 24. In essence, the left leg sleeve 23 and the right leg sleeve 24 are knitted as a single layer to provide a more breathable, flexible, and lightweight area to provide maximum comfort for the wearer. The lower left-leg hem 25 and the lower right-leg hem 26 may comprise an elastic band to hold the left leg sleeve 23 and the right leg sleeve 24 in place, preventing snagging and shifting when worn.

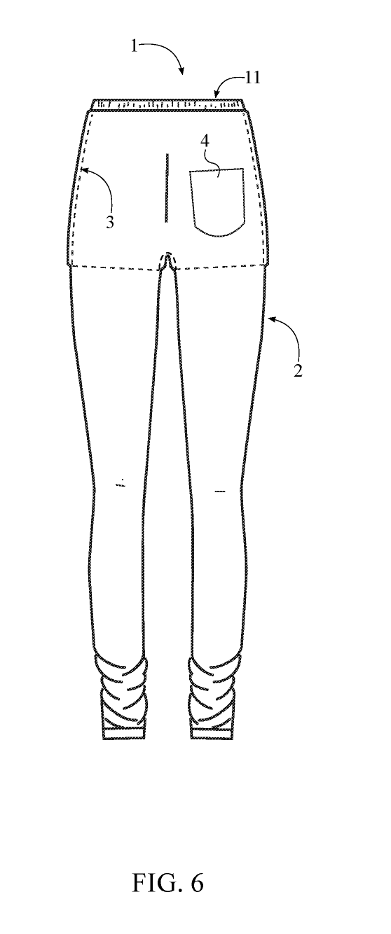

The present invention further comprises at least one pocket 4. As shown in FIG. 6-7, the pocket 4 is externally connected to the bifurcated legged outer layer 2 and adjacently positioned with the elastic rear portion 11. Since the pocket 4 is positioned normal to the bifurcated legged outer layer 2, the wearer can store items within the pocket 4. The present invention is preferably constructed from 100% merino wool or a merino and spandex blend. However, it is understood that like materials may be utilized in the construction of the present invention.

Although the invention has been explained in relation to its preferred embodiment, it is to be understood that many other possible modifications and variations can be made without departing from the spirit and scope of the invention as hereinafter claimed.

What is claimed is:

1. A pair of diaper cover leggings with high volume wet zone protection comprises:
a waistband;
a bifurcated legged outer layer;
an inner layer;
the waistband comprises an elastic rear portion and a drawstring front portion;
the bifurcated legged outer layer comprises a top outer edge, an outer crotch section, a left leg sleeve, and a right leg sleeve;
the elastic rear portion and the drawstring front portion being continuously connected to each other;
the top outer edge being perimetrically connected around the waistband;
the outer crotch section, the left leg sleeve, and the right leg sleeve being downwardly extended from the waistband;
a top inner edge of the inner layer being perimetrically connected around the waistband;
an inner crotch section of the inner layer being downwardly extended from the waistband to the outer crotch section; and
the inner crotch section being perimetrically connected to the outer crotch section.

2. The pair of diaper cover leggings with high volume wet zone protection as claimed in claim 1 comprises:
the top inner edge being adjacently positioned with the top outer edge; and
the top inner edge being perimetrically connected to the waistband through the bifurcated legged outer later.

3. The pair of diaper cover leggings with high volume wet zone protection as claimed in claim 1 comprises:
the inner crotch section comprises an upper left-leg opening and an upper right-leg opening;
the upper left-leg opening being adjacently connected within the left leg sleeve; and
the upper right-leg opening being adjacently connected within the right leg sleeve.

4. The pair of diaper cover leggings with high volume wet zone protection as claimed in claim 1 comprises:
the left leg sleeve being extended from the waistband to a lower left-leg hem of the bifurcated legged outer layer; and
the right leg sleeve being extended from the waistband to a lower right-leg hem of the bifurcated legged outer layer.

5. The pair of diaper cover leggings with high volume wet zone protection as claimed in claim 1 comprises:
at least one pocket;
the pocket being externally connected to the bifurcated legged outer layer; and
the pocket being adjacently positioned with the elastic rear portion.

6. The pair of diaper cover leggings with high volume wet zone protection as claimed in claim 1 comprises:
the drawstring front portion comprises a channel body, a left drawstring, and a right drawstring;
the channel body comprises a left end, a right end, and a drawstring opening;
the left end and the right end being connected to the elastic rear portion;
the drawstring opening being centrally positioned in between the left end and the right end;
the left drawstring being connected to the left end, extended along the channel body and through the drawstring opening; and
the right drawstring being connected to the right end, extended along the channel body and through the drawstring opening.

7. A pair of diaper cover leggings with high volume wet zone protection comprises:
a waistband;
a bifurcated legged outer layer;
an inner layer;
the waistband comprises an elastic rear portion and a drawstring front portion;
the bifurcated legged outer layer comprises a top outer edge, an outer crotch section, a left leg sleeve, and a right leg sleeve;
the elastic rear portion and the drawstring front portion being continuously connected to each other;
the top outer edge being perimetrically connected around the waistband;
the outer crotch section, the left leg sleeve, and the right leg sleeve being downwardly extended from the waistband;
a top inner edge of the inner layer being perimetrically connected around the waistband;
an inner crotch section of the inner layer being downwardly extended from the waistband to the outer crotch section;
the inner crotch section comprises an upper left-leg opening and an upper right-leg opening;
the upper left-leg opening being adjacently connected within the left leg sleeve; and
the upper right-leg opening being adjacently connected within the right leg sleeve.

8. The pair of diaper cover leggings with high volume wet zone protection as claimed in claim 7 comprises:
the top inner edge being adjacently positioned with the top outer edge; and
the top inner edge being perimetrically connected to the waistband through the bifurcated legged outer later.

9. The pair of diaper cover leggings with high volume wet zone protection as claimed in claim 7 comprises:
the left leg sleeve being extended from the waistband to a lower left-leg hem of the bifurcated legged outer layer; and
the right leg sleeve being extended from the waistband to a lower right-leg hem of the bifurcated legged outer layer.

10. The pair of diaper cover leggings with high volume wet zone protection as claimed in claim 7 comprises:
at least one pocket;
the pocket being externally connected to the bifurcated legged outer layer; and
the pocket being adjacently positioned with the elastic rear portion.

11. The pair of diaper cover leggings with high volume wet zone protection as claimed in claim 7 comprises:
the drawstring front portion comprises a channel body, a left drawstring, and a right drawstring;
the channel body comprises a left end, a right end, and a drawstring opening;
the left end and the right end being connected to the elastic rear portion;
the drawstring opening being centrally positioned in between the left end and the right end;
the left drawstring being connected to the left end, extended along the channel body and through the drawstring opening; and
the right drawstring being connected to the right end, extended along the channel body and through the drawstring opening.

12. A pair of diaper cover leggings with high volume wet zone protection comprises:
a waistband;
a bifurcated legged outer layer;
an inner layer;
at least one pocket;
the waistband comprises an elastic rear portion and a drawstring front portion;
the bifurcated legged outer layer comprises a top outer edge, an outer crotch section, a left leg sleeve, and a right leg sleeve;
the elastic rear portion and the drawstring front portion being continuously connected to each other;
the top outer edge being perimetrically connected around the waistband;
the outer crotch section, the left leg sleeve, and the right leg sleeve being downwardly extended from the waistband;
a top inner edge of the inner layer being perimetrically connected around the waistband;
an inner crotch section of the inner layer being downwardly extended from the waistband to the outer crotch section;
the inner crotch section comprises an upper left-leg opening and an upper right-leg opening;
the upper left-leg opening being adjacently connected within the left leg sleeve;
the upper right-leg opening being adjacently connected within the right leg sleeve;
the pocket being externally connected to the bifurcated legged outer layer; and
the pocket being adjacently positioned with the elastic rear portion.

13. The pair of diaper cover leggings with high volume wet zone protection as claimed in claim 12 comprises:
the top inner edge being adjacently positioned with the top outer edge; and
the top inner edge being perimetrically connected to the waistband through the bifurcated legged outer later.

14. The pair of diaper cover leggings with high volume wet zone protection as claimed in claim 12 comprises:
the left leg sleeve being extended from the waistband to a lower left-leg hem of the bifurcated legged outer layer; and
the right leg sleeve being extended from the waistband to a lower right-leg hem of the bifurcated legged outer layer.

15. The pair of diaper cover leggings with high volume wet zone protection as claimed in claim 12 comprises:
the drawstring front portion comprises a channel body, a left drawstring, and a right drawstring;
the channel body comprises a left end, a right end, and a drawstring opening;
the left end and the right end being connected to the elastic rear portion;
the drawstring opening being centrally positioned in between the left end and the right end;
the left drawstring being connected to the left end, extended along the channel body and through the drawstring opening; and
the right drawstring being connected to the right end, extended along the channel body and through the drawstring opening.
